(19) United States
(12) Patent Application Publication
(10) Pub. No.: US 2016/0005139 A1
(43) Pub. Date: Jan. 7, 2016

(54) METHOD FOR OPTIMIZING BLOOD UTILIZATION

(71) Applicant: Mediware Blood Management, LLC, Lenexa, KS (US)

(72) Inventor: Timothy Hannon, Carmel, IN (US)

(21) Appl. No.: 14/853,038

(22) Filed: Sep. 14, 2015

Related U.S. Application Data
(63) Continuation of application No. 12/257,003, filed on Oct. 23, 2008, now abandoned.

(51) Int. Cl. G06Q 50/22; G06Q 10/08

(52) U.S. Cl. CPC G06Q 50/22; G06Q 10/087

(57) ABSTRACT

A method for measuring the utilization of blood products including receiving blood product utilization data from a group of patients of a health care facility having related diagnoses over a specified period of time, executing a blood management software program to determine a patient population that received a blood product at the health care facility during the specified period of time, the patient population being identified from a group of patients having related diagnoses, generating a transfusion exposure score, analyzing a plurality of transfusion exposure scores, and transforming a transfusion exposure score from one of the plurality of identified patient populations into a transfusion propensity index score, the transfusion propensity index score being representative of a clinical opportunity to improve the utilization of blood products at the health care facility for which the transfusion exposure score of the identified patient population was calculated.

### RELATED APPLICATIONS

This application is a continuation of U.S. patent application Ser. No. 12/257,003 filed Oct. 23, 2008, the complete disclosure of which is hereby expressly incorporated in its entirety herein by this reference.

### TECHNICAL FIELD OF THE DISCLOSURE

The present invention generally relates to blood utilization systems, more particularly, to a method for optimizing blood utilization and to manage and forecast blood inventory.

### BACKGROUND

Transfusion of blood products is one of the most common interventions in a hospital setting. Examples of blood products are red blood cells, platelets, plasma and blood clotting agents. Twenty-nine million blood components are transfused each year, equating to nearly 80,000 blood components every day. Of great significance is the fact that a large portion of these blood products are not administered according to evidence-based practices, thereby consuming a precious resource without benefit to patients. Surprisingly, most physicians who order blood products lack formal training in transfusion therapy and most nursing schools generally fall short in their training for transfusion safety and blood administration competency. This lack of education and training complicates the decision to transfuse since it must be made in the context of an informed risk-to-benefit analysis. While it is true that through donor screening and testing the blood supply is the safest ever, blood transfusions are inherently dangerous and cause some degree of harm in every patient. Although the risk of viral transmission has been greatly reduced, the greatest risks to transfused patients are non-infectious hazards. Transfusion of blood products to the wrong patient (mistransfusion) is one of the leading causes of complications and death, along with transfusion related acute lung injury (TRALI) and transfusion associated circulatory overload (TACO). A growing body of evidence has also shown that blood transfusions correlate highly with increased rates of infection and poorer clinical outcomes because of transfusion related immunomodulation (TRIM). Despite these concerns, some physicians continue to over-utilize blood component therapy and order transfusions in a liberal fashion inconsistent with current scientific evidence. What has become increasingly obvious is that unnecessary transfusions are not only wasteful but are harmful and need to be avoided. Adding to these issues is the fact that blood utilization oversight is generally lacking as demonstrated by studies showing wide variations in transfusion practice between hospitals and among physicians at the same hospital. Additionally, there are concerns about legal liability for improper informed consent, inappropriate transfusions and transfusion-related adverse events. Finally, the costs of purchasing blood are increasing as well as the financial penalties for poor clinical outcomes related to inappropriate transfusion practices are increasing. The Centers for Medicare and Medicaid Services (CMS) and many commercial health insurance companies no longer pay for transfusion errors, bleeding complications in cardiac surgery, and a growing number of hospital acquired infections that are increased two to five-fold by blood transfusions.

In consideration of the preceding issues, there is a recognized need to develop systems to promote more appropriate blood utilization and to improve the quality, safety and efficiency of blood component therapy. Therefore, there is a need for a new method of measuring and assessing blood utilization to address the above shortcomings, concerns, and inefficiencies.

### SUMMARY OF THE INVENTION

The present teachings provide methods for analysis and benchmarking of hospitals to identify blood use reduction, cost savings opportunities, and to provide blood utilization forecasting and budgeting information.

In one form of the invention, a method of better managing blood product utilization is presented. The method comprises calculating a transfusion exposure score, the transfusion exposure score being the average amount of a blood product used for a patient population during a time period for a health care facility; calculating a mean transfusion exposure score, the mean transfusion exposure score being a geometric mean of a plurality of transfusion exposure scores within a database for the blood product and for the patient population over the period of time for a plurality of health care facilities; calculating a benchmark transfusion exposure score, the benchmark transfusion exposure score being the transfusion exposure score of a best practice facility having the lowest transfusion exposure score of the plurality of transfusion exposure scores in the database; calculating a transfusion propensity score, the transfusion propensity score being a ratio of the transfusion exposure score of the health care facility to the benchmark transfusion exposure score for the blood product and used for the patient population; and analyzing the transfusion exposure score, the mean transfusion exposure score, the benchmark transfusion exposure score, and the transfusion propensity score to quantify opportunities for operational and financial improvement within the health care provider facility.

In accordance with another form of the invention, a method for measuring the utilization of blood products comprises using a computing device to receive a data file from a health care facility, the data file including blood product utilization data obtained from a group of patients of the health care facility having related diagnoses over a specified period of time; executing, by a processor of the computing device, a blood management software program to determine a patient population from the data file that received a blood product at the health care facility during the specified period of time, the patient population being identified from the group of patients having related diagnoses; generating a transfusion exposure score by calculating, by the processor, a geometric mean of a plurality of quantified blood products used on the patient population during the specified period of time at the health care facility; analyzing, by the processor, a plurality of transfusion exposure scores for a plurality of identified patient populations from different health care facilities to determine a benchmark transfusion exposure score, wherein the plurality of identified patient populations each consist of a group of patients having related diagnoses, the benchmark transfusion exposure score representing a target blood product utilization index score for patients having related diagnoses; and transforming, by the processor, a transfusion exposure score from one of the plurality of identified patient populations produced by the blood management software program and the benchmark transfusion exposure score produced by the blood management software program into a transfusion propensity index score, the transfusion propensity index score being representative of a clinical opportunity to improve the utilization of blood products at the health care facility for which the transfusion exposure score of the identified patient population was calculated.

In accordance with yet another form of the invention, a method for measuring the utilization of blood products comprises receiving, with a computing device, a data file including blood product utilization data from a health care facility, the blood product utilization data being obtained from a group of patients of the health care facility having related diagnoses; executing, by a processor of the computing device, a blood management software program to identify a patient population from the data file that received a blood product at the health care facility over a specified period of time, the patient population being identified from the group of patients having related diagnoses; calculating, by the processor, a geometric mean of a plurality of quantified blood products used on the patient population during the specified period of time at the health care facility to generate a transfusion exposure score; analyzing, by the processor, a plurality of transfusion exposure scores for a plurality of identified patient populations from different health care facilities to determine a benchmark transfusion exposure score, wherein the plurality of identified patient populations each consist of a group of patients having related diagnoses, the benchmark transfusion exposure score representing a target blood product utilization index score for patients having related diagnoses; transforming, by the processor, a transfusion exposure score from one of the plurality of identified patient populations produced by the blood management software program and the benchmark transfusion exposure score produced by the blood management software program into a transfusion propensity index score, the transfusion propensity index score being representative of a clinical opportunity to improve the utilization of blood products at the health care facility for which the transfusion exposure score of the identified patient population was calculated; and analyzing, by the processor, the transfusion exposure score for one of the plurality of identified patient populations to generate a forecast model for assessing future utilization of blood products at the health care facility for which the transfusion exposure score of the identified patient population was calculated.

In accordance with still another form of the present invention, a method for measuring the utilization of blood products comprises using a computing device to receive a data file from a health care facility, the data file including blood product utilization data obtained from a group of patients of the health care facility having related diagnoses over a specified period of time; receiving the data file from a health care facility, the blood product utilization data being obtained from a group of patients of the health care facility having related diagnoses; executing, by a processor of a computing device, a blood management software program to identify a patient population from the data file that received a blood product at the health care facility during the specified period of time, the patient population being identified from the group of patients having related diagnoses; quantifying, by the processor, each blood product received by the patient population by considering at least one of the following variables: (a) individual medical procedures performed on the patient population; (b) individual diagnoses of the patient population; (c) physicians who treated the patient population and their respective medical specialties; and (d) the health care facility that treated the patient population; calculating, by the processor, a geometric mean of a plurality of quantified blood products used on the patient population during the specified period of time at the health care facility to generate a transfusion exposure score; analyzing, by the processor, a plurality of transfusion exposure scores from for a plurality of identified patient populations from different health care facilities to determine a benchmark transfusion exposure score, wherein the plurality of identified patient populations each consist of a group of patients having related diagnoses, the benchmark transfusion exposure score representing a target blood product utilization index score for patients having related diagnoses; transforming, by the processor, a transfusion exposure score from one of the plurality of identified patient populations produced by the blood management software program and the benchmark transfusion exposure score produced by the blood management software program into a transfusion propensity index score, the transfusion propensity index score being representative of a clinical opportunity to improve the utilization of blood products at the health care facility for which the transfusion exposure score of the identified patient population was calculated; and analyzing, by the processor, the transfusion exposure score for one of the plurality of identified patient populations to generate a forecast model for assessing future utilization of blood products at the health care facility for which the transfusion exposure score of the identified patient population was calculated.

### BRIEF DESCRIPTION OF THE DRAWINGS

The above-mentioned and other advantages of the present invention and the manner of obtaining them will become more apparent and the invention itself will be better understood by reference to the following description of the embodiments of the invention taken in conjunction with the accompanying drawings, wherein:

FIG. 1 is a schematic of data transfer from a health care provider to a data analysis facility;

FIG. 2 is a schematic of data integrity analysis; and

FIG. 3 is a schematic of data transfer from the data analysis facility to the health care provider.

### DETAILED DESCRIPTION

The embodiments of the present invention described below are not intended to be exhaustive or to limit the invention to the precise forms disclosed in the following detailed description. Rather, the embodiments are chosen and described so that others skilled in the art may appreciate and understand the principles and practices of the present invention.

According to the current teachings, methods involved in monitoring and improving the utilization of blood components may be altered in substantial ways. The method of the current teachings manipulates data obtained from health care provider facilities, such as hospitals and clinics. Patient-related data from the health care providers, hereinafter HCP, is obtained from these facilities and manipulated by a computer system as will be discussed in detail below. The computer system is located at a data analysis facility, hereinafter DAF, with provisions to review the data that is obtained from and sent to the HCP facilities as well as evaluating blood utilization and blood management practices. Therefore, according to the current teachings, patient data is accessed and processed at three different levels. First, the data is obtained at the HCP. Second, the data is logged and examined at the DAF. Third, the data is processed within the blood management system (hereinafter, BMS).

One goal of the method of the current teachings is to provide a blood utilization benchmarking and analysis package to a HCP. Such a package assists the HCP with internal and external benchmarking for the purpose of evaluating how blood is utilized at the HCP facility. Also, such a package will assist the HCP to identify blood product reduction and cost savings opportunities, and to provide blood utilization forecasting and budgeting information.

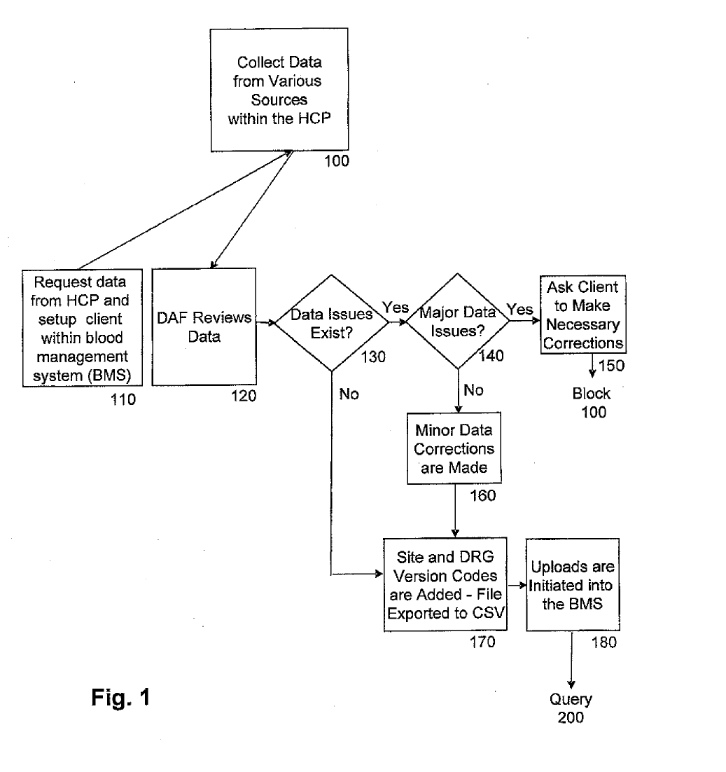

Referring to FIG. 1, a schematic of how data is transferred between a HCP and the DAF is presented. As indicated by block 100, data from various sources within HCP is collected. An example of the data is finance accounting data which contains all patient-related charges for the HCP products and services over a specified period of time. Another example of the collected data is blood product utilization and patient case volume for the same time period. This data includes blood utilization for patients who received blood or blood products as well patients who did not receive any blood products. This data is obtained from the HCP laboratory and finance departments.

As shown in FIG. 1, once the HCP data is collected, the data is transferred to the DAF for processing upon the DAF requesting the collected data, as seen by block 110. The data examination is accomplished either by personnel located at the DAF or by a pre-processing data analysis subsystem. Once the data is at the DAF, provisions exist to review the integrity of the data, as shown in block 120. The data is examined as indicated by the query 130. If there are issues with the data as entered in the HCP, the DAF examines whether the issues are major or minor. This query block has the reference numeral 140. If there are major issues associated with the data, the data is sent back to the HCP with a request for correction accompanied with information indicating the issues with the data as indicated by block 150. If, there are no major issues associated with the data, however, and there are minor issues associated with the data, then the DAF makes minor data corrections as indicated by block 160. An example of a major issue would be missing data. An example of a minor issue would be if the month was spelled out instead of in numeric form; for instance the word January instead of the value of 1. Once the data has been successfully examined, and corrected if necessary, site and diagnostic related groups, hereinafter, DRG, version codes are added to the data and the file is exported to the CSV, as indicated by block 170. CSV means “common separated value” and is a type of computer file that is used in the online database for data upload. The data is then uploaded to the BMS, as indicated by block 180. At this point the data processing departs the DAF and enters the BMS.

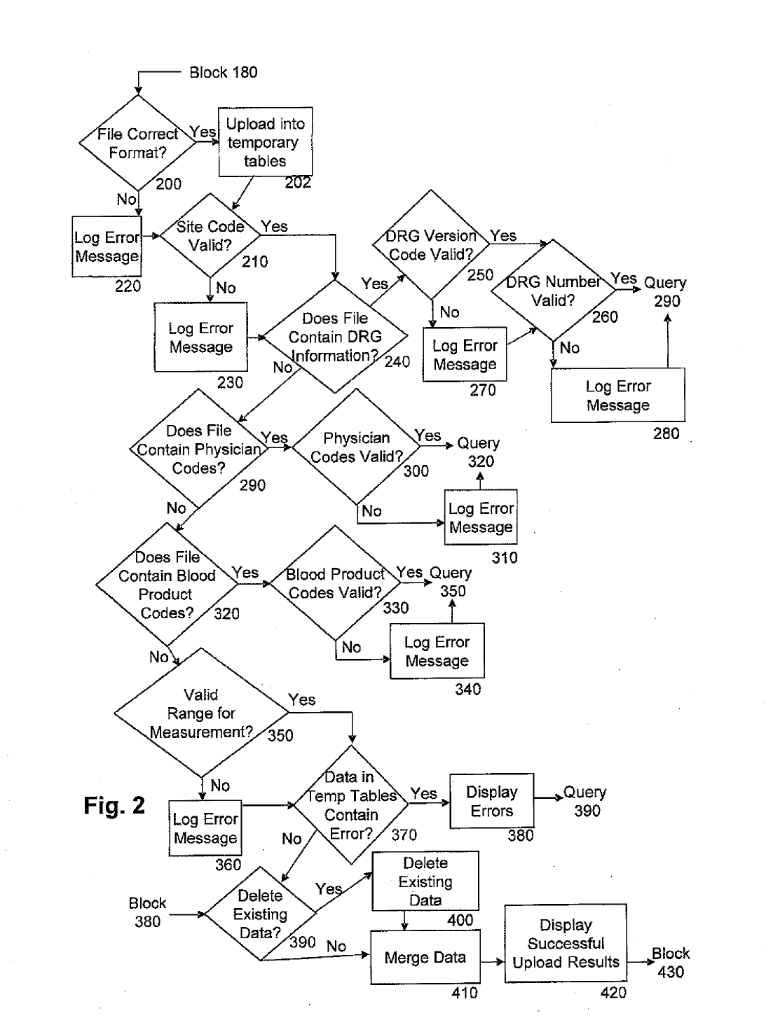

Referring to FIG. 2, once the data is imported to the BMS, initially certain housekeeping queries and actions are performed on the HCP data file, otherwise referred to as HCP data set. First, the BMS ascertains whether the file containing HCP patient data is formatted properly, as indicated by the query 200. If the HCP data file has the correct format, then the BMS uploads the file containing HCP patient data into temporary tables, as indicated by block 202. If, however, the HCP data file format is incorrect, an error message is logged by the BMS, as indicated by block 220. In either case, i.e., correct data format, BMS then ascertains whether the HCP data file contains a site code which is valid, as indicated by query 210. If the format is correct, BMS then continues to the query 210. If the HCP data file has a valid site code, then the BMS ascertains whether the HCP data file contains DRG information, as indicated by query 240. If, however, the HCP data file does not contain a valid site code, an error message is logged by the BMS, as indicated by block 230. BMS then continues to the query 240. DRG is related to a collection of individual diagnoses that are related. DRGs are common groups of patients that were defined by the government for the purpose of billing. There a total of 500 DRGs. For example, DRG 174 is gastrointestinal hemorrhage with complications. If the HCP data file contains DRG information, then the BMS ascertains whether the DRG version code is valid, as indicated by query 250. If the HCP data file has a valid DRG version code, then the BMS ascertains whether the DRG numbers are valid, as indicated by query 260. If, however, the DRG codes are not valid, an error message is logged by the BMS, as indicated by block 270. BMS then continues to the query 260. If DRG numbers are valid, then the BMS continues to query 290. If, however, the DRG numbers are not valid, an error message is logged by the BMS, as indicated by block 280. BMS then continues to the query 290. Referring back to query 240, if the HCP data file does not contain DRG information, the BMS then continues with query 290.

In Query 290 the BMS determines whether the HCP data file contains physician codes. If yes, the BMS then ascertain whether the physician codes are valid, as indicated by query 300. If the physician codes are valid, the BMS then proceeds to query 320. If, however, the physician codes are not valid, an error message is logged by the BMS, as indicated by block 310. BMS then continues to the query 320. Also, if the HCP data file does not contain physician codes, the BMS continued to query 320.

In Query 320 the BMS determines whether the HCP data file contain blood product codes. If the answer is yes, the BMS proceeds to query 330. In query 330, the BMS determines whether the blood product codes are valid. If the blood product codes are valid, the BMS proceeds to query 350. If, however, the blood product codes are not valid, an error message is logged by the BMS, as indicated by block 340. BMS then continues to the query 350. Also, if the HCP data file does not contain blood product codes, the BMS continued to query 350.

In Query 350 the BMS determines whether the HCP data file contains a valid range for measurements. If yes, then the BMS proceeds to query 370. If the HCP data file does not contain a valid range for measurements, an error message is logged by the BMS, as indicated by block 360. The BMS then proceeds to query 370. In query 370, the BMS finds out whether the HCP data uploaded into temporary tables contains error. An Example of an error would be blanks in the data file where there should be a value. If the HCP data file does not contain errors, then the BMS determines whether the existing data should be deleted or overwritten, as shown by query 390. If the HCP data file contains errors, then BMS displays the errors as indicated by block 380 before proceeding to query 390. If the answer to query 390 is yes, the BMS deletes or overwrites the existing data as indicated by block 400. The BMS then proceeds to merging the data, as indicated by block 410. If, however, the answer to query 390 is no, the BMS then proceeds to block 410. The data files maybe iteratively uploaded, and thus these files are merged. Examples of these iterative data file uploads are physician listing and blood product utilization files. The BMS then proceeds to displaying successful upload results of the HCP data file, as indicated by block 420. At this point, after passing the above housekeeping error checks, the HCP data files have been added to the Structured Query Language (SQL) database.

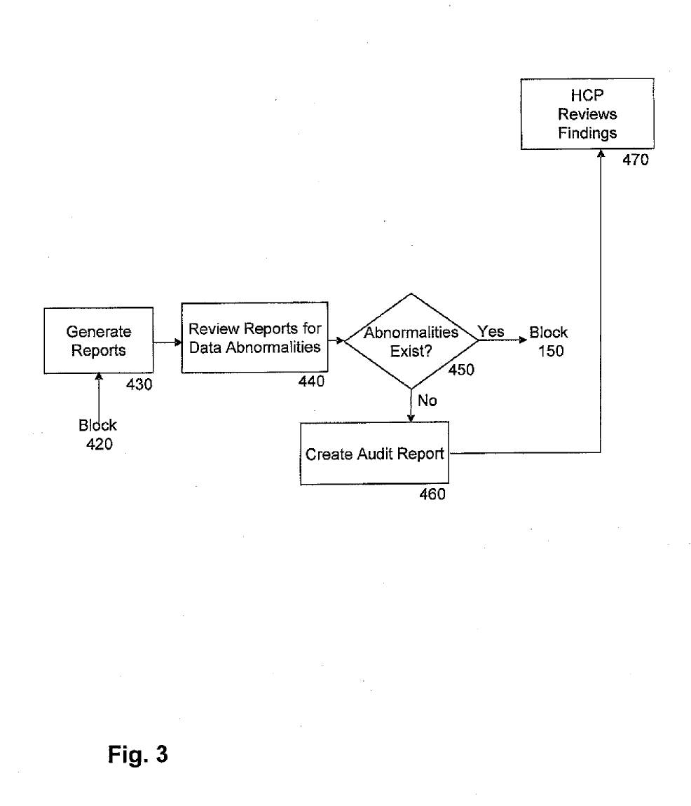

Now referring to FIG. 3, BMS generates reports for the DAF, as indicated by block 430. These reports are listed in Appendix A. The DAF reviews the reports for data abnormalities, as indicated by block 440. Examples of data abnormalities are labeling that is incomplete or headers and footers which are not complete. If data abnormalities exist, the DAF proceeds to Block 150. If, however, no data abnormalities exist, DAF creates an audit report, as indicated in block 460, which is a compilation of the reports into one comprehensive grouping. The HCP then reviews the audit report, as indicated by block 470.

The foregoing discussion relates to housekeeping tasks associated with importing the HCP data set. We now turn to the substantial portion of how the BMS treats the HCP data set.

The BMS begins by searching the HCP data file to identify patients who received blood products during their episodes of care. Blood related charges for the major blood products are obtained and “mapped” to the unique codes employed by the HCP.

After identifying the patients in the HCP data file who have received blood products, a series of sorts are then performed to identify and quantify blood utilization for each type of blood product by a number of variables, including DRG, principle procedural code, physician specialty, and individual physicians. For each patient the data includes the discharge date, DRG number, version of DRG, attending doctor by code, principle procedure surgeon by code, number of units given of plasma, platelets, autologous blood, cryoprecipitate, packed red blood cells, whole blood, length of stay, and total charges for the stay.

A transfusion exposure score (hereinafter, TES) is a unit used for comparing blood transfusion data analysis and is used directly or indirectly in most of the analyses described below. These teachings utilize several associated TES parameters. These are Index HCP TES, Mean TES, and Benchmark TES. In general, the TES is the average amount of a particular blood product, expressed in units of blood, used for a specified patient population during a specified time period. The specified patient population is either from the same DRG or same principle procedure code. It is important to note that this average amount includes all patients in the specified population, e.g., both those patients who received blood products as well as those who did not. Since the TES value is derived by the same methodology each time, it allows valid comparisons of blood utilization within a HCP over time as well as among different HCPs during similar time periods. Of particular value is the comparison of the index HCP, i.e., the HCP under investigation, TES performance to comparable HCPs such as those with similar case mix index, i.e., those with similar specialty services such as level I trauma or organ transplant, community vs. academic hospital, or other hospitals within certain groups such as health systems or consortiums. In general, lower TES values indicate more optimal blood utilization at a particular HCP.

The BMS derives a mean TES (hereinafter, MTES) using a novel method which is a geometric mean of the TES values within the BMS database for a specific blood product and for a specific patient population over a specified period of time. The purpose of MTES is to give client HCP a comparison to average blood utilization as an indicator of a HCP performance for any specific blood product and patient population during a given time period. A geometric means removes the outlier values from the selected database parameters which gives a truer picture of the actual results. In one embodiment, data points beyond three standard deviation away from the mean in a Gaussian distribution are discarded and a new mean value is generated, i.e., the MTES. All data for the time frame selected are used to derive the geometric mean value. HCP data is analyzed over a 12 month period and subsequent evaluations use a moving twelve month average.

BMS also derives a benchmark TES (hereinafter, BTES) for each patient population from “best practice facilities” within the proprietary DAF database. The purpose of the BTES is to provide a comparison and a possible target utilization rate for the client HCP. The BTES derivation utilizes a three step screening process. These are: 1) overall lowest TES value for a specific patient population (typically a DRG) is determined using a screening tool to identify a benchmark candidate organization, which identifies organizations which utilize blood resources in an efficient manner; 2) a minimum annual case volume is required to validate that the organization performs a sufficient number of cases to be proficient, (provisions are in place so that case volumes can be modified for particularly “niche” DRGs or principle procedure codes that are only performed in specialty centers); and 3) if the benchmark candidate passes the first two screens, then the patient population of interest for that organization must meet or exceed average patient outcome metrics, such as complications, length of stay and mortality rates, as can be determined from publically available sources such as the Center for Medicare and Medicaid Services (CMS) Hospital Compare website (See http://www.hospitalcompare.hhs.gov) or the MedPar database. In one embodiment the minimum number of cases for an organization to be proficient is about 52 per year, i.e., one per week. In another embodiment the minimum number is about 30 per year.

The BMS then derives a transfusion propensity score (hereinafter, TPS) from the relationship of the index HCP TES for a specific blood product for a specific patient population over a specified period of time to the corresponding BTES. The purpose of the TPS is to provide a ratio of index HCP blood utilization to benchmark utilization for specific blood products and specific patient populations (HCP TES/BTES). For example, a TPS of 1.5 for a particular blood product and for a particular DRG would indicate blood utilization by the index HCP that is 50% greater than the benchmark rate of utilization, while a TPS of 2.0 would indicate 100% greater utilization than the benchmark. This TPS score can be used to target and prioritize efforts to improve blood utilization by specific blood product type, e.g., red blood cells vs. plasma. The TPS score can also be used to target and prioritize efforts to improve blood utilization by patient population, e.g., DRG 105 vs. 209. Alternatively, TPS can be used to improve blood utilization based on physician specialty, e.g., cardiac surgery vs. orthopedic surgery. The TPS can also be used to readily track utilization trends for a client HCP, both internally among the different departments of the HCP or by comparing a client HCP to the benchmark, over time.

The client HCP can utilize the relationship between the client HCP index TES and the corresponding BTES as a way to quantify opportunities for operational and financial improvement to achieve and to surpass benchmark rates of utilization. The purpose of this quantification is to provide hospital decision makers the scope and scale of blood utilization opportunities for forecasting and business case analysis. Further detail on maximizing blood utilization opportunities are provided below.

The blood product unit savings opportunity for a specific blood product and for a specific patient population over a specified period of time is derived from the difference between the index HCP TES and the corresponding BTES. This difference is the unit savings opportunity per case, from which a total unit savings opportunity can be derived by factoring the number of cases performed during a specified period of time. From this blood product unit savings opportunity, the financial opportunity for savings is developed by incorporating the current “actual cost” of blood products for the client HCP. The actual cost calculations incorporates both the cost savings from purchasing of the blood product and the transfusion related costs such as labor, supplies and allocated overhead. Also, the cost calculation incorporates costs connected with transfusion associated adverse events. By using the actual cost of blood products for the client hospital over a specified period of time, a business case analysis can be derived for cost opportunities to target and prioritize efforts to improve blood utilization by blood product type, e.g., red blood cells vs. plasma; or by patient population, e.g., DRG 105 vs. 209; or by physician group, e.g., cardiac surgery vs. orthopedic surgery. This cost data is also used for return on investment (ROI) calculations that can be used in business case analyses. Therefore, the calculations associated with the actual cost of blood transfusion allows decision makers in the HCP to understand full breadth of how much blood transfusion is actually costing their facility. This understanding can assist these decision makers in developing more accurate blood utilization oversight and operational business models. For example, from a financial point of view, using the blood product unit savings opportunity, a decision maker can decide whether to purchase a piece of equipment to improve the utilization of the blood products as part of the ROI calculations.

The index HCP TES is also used for budgeting and blood inventory forecasting. For example, since the TES represents average utilization of a blood product for a specified patient population, such as a DRG, a forecast can be made for the impact on blood utilization and blood costs due to increases or decreases in general patient volumes or for specific patient populations. For example, if a client HCP was contemplating increasing orthopedic joint replacement patient volumes by building a new surgery center and recruiting more patients, blood utilization per case information would lead to projections for blood utilization and blood costs. Also, this information would be useful to the local blood supplier to help meet an increased demand. More particularly, if the HCP MTES data is available, the MTES data is utilized to forecast the number of blood products needed based on the forecasted number of procedure in the new surgery center for unique DRGs.

Additionally, forecasting using the index HCP TES can be used to improve blood supply inventory and peak demand within a community. This is because blood product utilization by a particular procedure or DRG can be linked with surgical or medical case volumes on a monthly, weekly or even daily basis. Demand forecasting to include elective surgery schedules can then help predict the need for donor recruitment in a more “just-in-time” manner to reduce inventory needs at both the blood collection center and client HCP. This can lead to less donor demand, fewer blood product outdates and lower on-hand inventory costs.

Forecasting using the index TES from a HCP or a group of HCPs can also be used for disaster planning scenarios or blood shortage scenarios. For example, the impact of increased blood demand for specific injuries such as radiation exposure from a “dirty bomb” resulting in victims with bone marrow suppression can be estimated using index hospital TES information. Also the current teachings can be used to calculate the effectiveness of interventions to reduce blood demand, e.g., cancellation of elective surgical procedures at the index HCP or HCP within a community in response to a disaster which would be accomplished using the corresponding TES information at these HCPs.

While exemplary embodiments incorporating the principles of the present invention have been disclosed hereinabove, the present invention is not limited to the disclosed embodiments. Instead, this application is intended to cover any variations, uses, or adaptations of the invention using its general principles. Further, this application is intended to cover such departures from the present disclosure as come within known or customary practice in the art to which this invention pertains and which fall within the limits of the appended claims.

### APPENDIX A

### Listing of BloodStat Reports

Autologous RBC Utilization by Calendar Year

BloodStat™ Scorecard without Restriction

BloodStat™ Scorecard

Cryoprecipitate Utilization by Calendar Year

Cryoprecipitate per Acute Discharge by Calendar Year

Monthly Detail for Cryoprecipitate per Acute Discharge

FFP Utilization by Calendar Year

FFP per Acute Discharge by Calendar Year

Monthly Detail for FFP per Acute Discharge

RBC Utilization by Calendar Year

RBC per Acute Discharge by Calendar Year

Monthly Detail for RBC per Acute Discharge

Platelets Utilization by Calendar Year

Platelets per Acute Discharge by Calendar Year

Monthly Detail for Platelets per Acute Discharge

Overall Cost

Patients with Blood Usage

Reduction Outcomes

Top 15 DRGs by Total Blood Products

Transfusion Cost Data

Transfusion Use Data

Use by Physicians

Find Lowest TES Scores

Diagnostic Related Groups and Codes

What is claimed is:

1. A method for measuring the utilization of blood products, comprising:
using a computing device to receive a data file from a health care facility, the data file including blood product utilization data obtained from a group of patients of the health care facility having related diagnoses over a specified period of time;
executing, by a processor of the computing device, a blood management software program to determine a patient population from the data file that received a blood product at the health care facility during the specified period of time, the patient population being identified from the group of patients having related diagnoses;
generating a transfusion exposure score by calculating, by the processor, a geometric mean of a plurality of quantified blood products used on the patient population during the specified period of time at the health care facility;
analyzing, by the processor, a plurality of transfusion exposure scores for a plurality of identified patient populations from different health care facilities to determine a benchmark transfusion exposure score, wherein the plurality of identified patient populations each consist of a group of patients having related diagnoses, the benchmark transfusion exposure score representing a target blood product utilization index score for patients having related diagnoses; and
transforming, by the processor, a transfusion exposure score from one of the plurality of identified patient populations produced by the blood management software program and the benchmark transfusion exposure score produced by the blood management software program into a transfusion propensity index score, the transfusion propensity index score being representative of a clinical opportunity to improve the utilization of blood products at the health care facility for which the transfusion exposure score of the identified patient population was calculated.

2. The method of claim 1, further comprising verifying the integrity of the blood product utilization data by examining it with a computer operated data analysis subsystem.

3. The method of claim 1, further comprising quantifying each blood product received by the patient population by considering at least one of the following variables:
(a) individual medical procedures performed on the patient population;
(b) individual diagnoses of the patient population;
(c) physicians who treated the patient population and their respective medical specialties; and
(d) the health care facility that treated the patient population.

4. The method of claim 1, further comprising analyzing the transfusion exposure score for one of the plurality of identified patient populations to generate a forecast model for assessing future utilization of blood products at the health care facility for which the transfusion exposure score of the identified population was calculated.

5. The method of claim 1, wherein the benchmark transfusion exposure score represents at least one of the following variables:
(a) a health care facility having a lowest calculated transfusion exposure score for a specific patient population;
(b) a minimum number of medical cases performed by the health care facility on the patient population each year; and
(c) a minimum patient outcome threshold for the patient population.

6. The method of claim 5, wherein the minimum number of medical cases performed by the health care facility on the patient population each year is at least about 52.

7. The method of claim 5, wherein the minimum number of medical cases performed by the health care facility on the patient population each year is at least about 30.

8. The method of claim 1, wherein the step of identifying the patient population from the data file that received the blood product comprises identifying patients who received at least one of red blood cells, plasma, platelets, cryoprecipitate, autologous blood and whole blood.

9. A method for measuring the utilization of blood products, comprising:
receiving, with a computing device, a data file including blood product utilization data from a health care facility, the blood product utilization data being obtained from a group of patients of the health care facility having related diagnoses;
executing, by a processor of the computing device, a blood management software program to identify a patient population from the data file that received a blood product at the health care facility over a specified period of time, the patient population being identified from the group of patients having related diagnoses;
calculating, by the processor, a geometric mean of a plurality of quantified blood products used on the patient population during the specified period of time at the health care facility to generate a transfusion exposure score;
analyzing, by the processor, a plurality of transfusion exposure scores for a plurality of identified patient populations from different health care facilities to determine a benchmark transfusion exposure score, wherein the plurality of identified patient populations each consist of a group of patients having related diagnoses, the benchmark transfusion exposure score representing a target blood product utilization index score for patients having related diagnoses;
transforming, by the processor, a transfusion exposure score from one of the plurality of identified patient populations produced by the blood management software program and the benchmark transfusion exposure score produced by the blood management software program into a transfusion propensity index score, the transfusion propensity index score being representative of a clinical opportunity to improve the utilization of blood products at the health care facility for which the transfusion exposure score of the identified patient population was calculated; and
analyzing, by the processor, the transfusion exposure score for one of the plurality of identified patient populations to generate a forecast model for assessing future utilization of blood products at the health care facility for which the transfusion exposure score of the identified patient population was calculated.

10. The method of claim 9, further comprising verifying the integrity of the blood product utilization data by examining it with a computer operated data analysis subsystem.

11. The method of claim 9, wherein the benchmark transfusion exposure score represents at least one of the following variables:
(a) a health care facility having a lowest calculated transfusion exposure score
(b) for a specific patient population;
(c) a minimum number of medical cases performed by the health care facility on the patient population each year; and
a minimum patient outcome threshold for the patient population.

12. The method of claim 11, wherein the minimum number of medical cases performed by the health care facility on the patient population each year is at least about 52.

13. The method of claim 11, wherein the minimum number of medical cases performed by the health care facility on the patient population each year is at least about 30.

14. The method of claim 9, further comprising quantifying each blood product received by the patient population by considering at least one of the following variables:
(a) individual medical procedures performed on the patient population;
(b) individual diagnoses of the patient population;
(c) physicians who treated the patient population and their respective medical specialties; and
(d) the health care facility that treated the patient population.

15. The method of claim 9, wherein the step of identifying the patient population from the data file that received the blood product comprises identifying patients who received at least one of red blood cells, plasma, platelets, cryoprecipitate, autologous blood and whole blood.

16. A method for measuring the utilization of blood products, comprising:
using a computing device to receive a data file from a health care facility, the data file including blood product utilization data obtained from a group of patients of the health care facility having related diagnoses over a specified period of time;
receiving the data file from a health care facility, the blood product utilization data being obtained from a group of patients of the health care facility having related diagnoses;
executing, by a processor of a computing device, a blood management software program to identify a patient population from the data file that received a blood product at the health care facility during the specified period of time, the patient population being identified from the group of patients having related diagnoses;
quantifying, by the processor, each blood product received by the patient population by considering at least one of the following variables:
(a) individual medical procedures performed on the patient population;
(b) individual diagnoses of the patient population;
(c) physicians who treated the patient population and their respective medical specialties; and
(d) the health care facility that treated the patient population;
calculating, by the processor, a geometric mean of a plurality of quantified blood products used on the patient population during the specified period of time at the health care facility to generate a transfusion exposure score;
analyzing, by the processor, a plurality of transfusion exposure scores from for a plurality of identified patient populations from different health care facilities to determine a benchmark transfusion exposure score, wherein the plurality of identified patient populations each consist of a group of patients having related diagnoses, the benchmark transfusion exposure score representing a target blood product utilization index score for patients having related diagnoses;
transforming, by the processor, a transfusion exposure score from one of the plurality of identified patient populations produced by the blood management software program and the benchmark transfusion exposure score produced by the blood management software program into a transfusion propensity index score, the transfusion propensity index score being representative of a clinical opportunity to improve the utilization of blood products at the health care facility for which the transfusion exposure score of the identified patient population was calculated; and
analyzing, by the processor, the transfusion exposure score for one of the plurality of identified patient populations to generate a forecast model for assessing future utilization of blood products at the health care facility for which the transfusion exposure score of the identified patient population was calculated.

17. The method of claim 16, further comprising verifying the integrity of the blood product utilization data by examining it with a computer operated data analysis subsystem.

18. The method of claim 16, wherein the benchmark transfusion exposure score represents at least one of the following variables:
(a) a health care facility having a lowest calculated transfusion exposure score for a specific patient population;
(b) a minimum number of medical cases performed by the health care facility on the patient population each year; and
(c) a minimum patient outcome threshold for the patient population.

19. The method of claim 18, wherein the minimum number of medical cases performed by the health care facility on the patient population each year is at least about 30.

20. The method of claim 16, wherein the step of identifying the patient population from the data file that received the blood product comprises identifying patients who received at least one of red blood cells, plasma, platelets, cryoprecipitate, autologous blood and whole blood.
